(19) United States
(12) Patent Application Publication
(10) Pub. No.: US 2010/0043095 A1
(43) Pub. Date: Feb. 18, 2010

(54) PRODUCTION, FORMULATION, AND USES OF STABLE LIQUID HARPIN PROTEIN FORMULATIONS

(75) Inventor: Zhongmin Wei, Kirkland, WA (US)

Correspondence Address: NIXON PEABODY LLP - PATENT GROUP, 1100 CLINTON SQUARE, ROCHESTER, NY, 14604, US

(73) Assignee: PLANT HEALTH CARE, INC., Pittsburgh, PA (US)

(21) Appl. No.: 12/536,750

(22) Filed: Aug. 6, 2009

Related U.S. Application Data
(60) Provisional application No. 61/088,195, filed on Aug. 12, 2008.

(51) Int. Cl. A01H 1/06; A01N 37/18; A61K 9/12; A61K 9/00

(52) U.S. Cl. 800/276; 514/12; 424/45; 424/400

(57) ABSTRACT

The present invention relates to a method of making a stable liquid composition containing a harpin protein or polypeptide. Also disclosed is a composition comprising an aqueous carrier, a harpin protein or polypeptide, an effective amount of a biocidal agent, and optionally, an effective amount of one or both of a protease inhibitor and a non-ionic surfactant. The composition retains harpin activity for at least about 72 hours. The present invention also relates to a method of inducing a plant response by applying to a plant or a plant seed the composition of the present invention.

This application claims the priority benefit of U.S. Provisional Patent Application Ser. No. 61/088,195, filed Aug. 12, 2008, which is hereby incorporated by reference in its entirety.

### FIELD OF THE INVENTION

This invention relates to the production, formulation, and use of stable liquid harpin protein formulations.

### BACKGROUND OF THE INVENTION

Plants have evolved a complex array of biochemical pathways that enable them to recognize and respond to environmental signals, including pathogen infection. There are two major types of interactions between a pathogen and plant-compatible and incompatible. When a pathogen and a plant are compatible, disease generally occurs. If a pathogen and a plant are incompatible, the plant is usually resistant to that particular pathogen. In an incompatible interaction, a plant will restrict pathogen proliferation by causing localized necrosis, or death of tissues, to a small zone surrounding the site of infection. This reaction by the plant is defined as the hypersensitive response (“HR”) (Kiraly, “Defenses Triggered by the Invader: Hypersensitivity,” Plant Disease: An Advanced Treatise 5:201-224 J. G. Horsfall and E. B. Cowling, eds. Academic Press, New York (1980); Klement, “Hypersensitivity,” Phytopathogenic Prokaryotes 2:149-177, M. S. Mount and G. H. Lacy, eds. Academic Press, New York (1982)). The localized cell death not only contains the infecting pathogen from spreading further but also leads to a systemic resistance preventing subsequent infections by other pathogens. Therefore, HR is a common form of plant resistance to diseases caused by bacteria, fungi, nematodes, and viruses.

A set of genes designated as hrp (Hypersensitive Response and Pathogenicity) is responsible for the elicitation of the HR by pathogenic bacteria, including, among others, Erwinia spp, Pseudomonas spp, Xanthomonas spp, and Ralstonia spp (Willis et al. “hrp Genes of Phytopathogenic Bacteria,” Mol. Plant-Microbe Interact. 4:132-138 (1991); Bonas, “hrp Genes of Phytopathogenic Bacteria,” pp. 79-98 in: Current Topics in Microbiology and Immunology, Vol. 192, Bacterial Pathogenesis of Plants and Animals: Molecular and Cellular Mechanisms. J. L. Dangl, ed. Springer-Verlag, Berlin (1994); Alfano et al., “Bacterial Pathogens in Plants: Life Up Against the Wall,” Plant Cell 8:1683-98 (1996)). Typically, there are multiple hrp genes clustered in a 30-40 kb segment of DNA. Mutation in any one of the hrp genes will result in the loss of bacterial pathogenicity in host plants and the HR in non-host plants.

On the basis of genetic and biochemical characterization, the function of the hrp genes can be classified into three groups: (1) structural genes encoding extracellularly located HR elicitors, for example, harpins (Wei et al. “Harpin, Elicitor of the Hypersensitive Response Produced by the Plant Pathogen Erwinia amylovora,” Science 257:85 (1992); He et al, “Pseudomonas syringae pv. Syringae harpinₚₛₛ: A Protein that is Secreted Via the Hrp Pathway and Elicits the Hypersensitive Response in Plants,” Cell 73:1255 (1993); Arlat et al. “PopA1, a Protein which Induces a Hypersensitive-Like Response on Specific Petunia Genotypes, Is Secreted via the Hrp Pathway of Pseudomonas solanacearum,” EMBO J. 13:543-53 (1994); Kim et al., “HrpW of Erwinia amylovora, a New Harpin that Contains a Domain Homologous to Pectate Lyases of a Distinct Class,” J. Bacteriol. 180:5203-10 (1998)); (2) secretion genes encoding a secretory apparatus for exporting HR elicitors and other proteins from the bacterial cytoplasm to the cell surface or extracellular space (Van Gijsegem et al., “Evolutionary Conservation of Pathogenicity Determinants Among Plant and Animal Pathogenic Bacteria,” Trends Microbiol. 1:175-180 (1993); He et al, “Pseudomonas syringae pv. Syringae harpinₚₛₛ: A Protein that is Secreted Via the Hrp Pathway and Elicits the Hypersensitive Response in Plants,” Cell 73:1255 (1993); Wei et al., “HrpI of Erwinia amylovora Functions in Secretion of Harpin and is a Member of a New Protein Family,” J. Bacteriol. 175:7985-67 (1993); Arlat et al. “PopA1, a Protein which Induces a Hypersensitive-Like Response on Specific Petunia Genotypes, Is Secreted via the Hrp Pathway of Pseudomonas solanacearum,” EMBO J. 13:543-53 (1994); Galan et al., “Cross-talk Between Bacterial Pathogens and Their Host Cells,” Ann. Rev. Cell Dev. Biol. 12:221-55 (1996); Bogdanove et al., “Erwinia amylovora Secretes Harpin via a Type III Pathway and Contains a Homolog of yopN of Yersinia,” J. Bacteriol. 178:1720-30 (1996); Bogdanove et al., “Homology and Functional Similarity of a hrp-linked Pathogenicity Operon, dspEF, of Erwinia amylovora and the avrE Locus of Pseudomonas syringae Pathovar Tomato,” Proc. Natl. Acad. Sci. USA 95:1325-30 (1998)); and (3) regulatory genes that control the expression of hrp genes (Wei, “Harpin, Elicitor of the Hypersensitive Response Produced by the Plant Pathogen Erwinia amylovora,” Science 257:85 (1992); Wei et al., “hrpL Activates Erwinia amylovora hrp Genes in Response to Environmental Stimuli,” J. Bacteriol. 174:1875-82 (1995); Xiao et al., “A Single Promoter Sequence Recognized by a Newly Identified Alternate Sigma Factor Directs Expression of Pathogenicity and Host Range Determinants in Pseudomonas syringae,” J. Bacteriol. 176:3089-91 (1994); Kim et al., “The hrpA and hrpC Operons of Erwinia amylovora Encode Components of a Type III Pathway that Secretes Harpin,” J. Bacteriol. 179:1690-97 (1997); Kim et al., “HrpW of Erwinia amylovora, a New Harpin that Contains a Domain Homologous to Pectate Lyases of a Distinct Class,” J. Bacteriol. 180:5203-10 (1998); Wengelnik et al., “HrpG, A Key hrp Regulatory Protein of Xanthomonas campestris pv. Vesicatoria is Homologous to Two Component Response Regulators,” Mol. Plant-Microbe Interact. 9:704-12 (1996)). Because of their role in interactions between plants and microbes, hrp genes have been a focus for bacterial pathogenicity and plant defense studies.

In addition to the local defense response, HR also activates the defense system in uninfected parts of the same plant. This results in a general systemic resistance to a secondary infection termed Systemic Acquired Resistance (“SAR”) (Ross, “Systemic Acquired Resistance Induced by Localized Virus Infections in Plants,” Virology 14:340-58 (1961); Malamy et al., “Salicylic Acid and Plant Disease Resistance,” Plant J. 2:643-654 (1990)). SAR confers long-lasting systemic disease resistance against a broad spectrum of pathogens and is associated with the expression of a certain set of genes (Ward et al., “Coordinate Gene Activity in Response to Agents that Induce Systemic Acquired Resistance,” Plant Cell 3:1085-94 (1991)). SAR is an important component of the disease resistance of plants and has long been of interest, because the potential of inducing the plant to protect itself could significantly reduce or eliminate the need for chemical pesticides. SAR can be induced by biotic (microbes) or abiotic (chemical) agents (Gorlach et al., “Benzothiadiazole, A Novel Class of Inducers of Systemic Acquired Resistance, Activates Gene Expression and Disease Resistance In Wheat,” Plant Cell 8:629-43 (1996)). Historically, weak virulent pathogens were used as a biotic inducing agent for SAR. Non-virulent plant growth promotion bacteria were also reported to be able to induce resistance of some plants against various diseases.

Biotic agent-induced SAR has been the subject of much research. With the advancement of molecular biology, the first proteinaceous HR elicitor with broad host spectrum was isolated in 1992 from Erwinia amylovora, a pathogenic bacterium causing fire blight in apple and pear. The HR elicitor was named “harpin” and is now known as harpin_{Ea} or HrpN_{Ea}. Harpin_{Ea} consists of 403 amino acids with a molecular weight about 40 kDa. The gene encoding this protein, hrpN, is contained in a 1.3 kb DNA fragment located in the middle of the hrp gene cluster. Harpin_{Ea} is secreted into the extracellular space and is very sensitive to proteinase digestion.

Since harpin_{Ea} was isolated from Erwinia amylovora, several other harpins or harpin-like proteins have been isolated from other major groups of plant pathogenic bacteria. In addition to harpin_{Ea}, the following harpin or harpin-like proteins have been isolated and characterized: HrpN of Erwinia chrysanthemi, Erwinia carotovora (Wei et al., “Harpin, Elicitor of the Hypersensitive Response Produced by the Plant Pathogen Erwinia amylovora,” Science 257:85 (1992)), and Erwinia stewartii; HrpZ of Pseudomonas syringae (He et al., “Pseudomonas syringae pv. Syringae harpinₚₛₛ: A Protein that is Secreted Via the Hrp Pathway and Elicits the Hypersensitive Response in Plants,” Cell 73:1255 (1993)), PopA of Ralstonia solanacearum (Arlat et al. “PopA1, a Protein which Induces a Hypersensitive-Like Response on Specific Petunia Genotypes, Is Secreted via the Hrp Pathway of Pseudomonas solanacearum,” EMBO J. 13:543-53 (1994)); and HrpW of Erwinia amylovora (Kim et al., “HrpW of Erwinia amylovora, a New Harpin that Contains a Domain Homologous to Pectate Lyases of a Distinct Class,” J. Bacteriol. 180:5203-10 (1998)) and Pseudomonas syringae.

Harpin-like proteins share common characteristics. They are heat-stable and glycine-rich proteins with not more than one cysteine residue (more typically, no cysteine residues), sensitive to digestion by proteinases, and elicit the HR and induce resistance in many plants against many diseases. Based on their shared biochemical and biophysical characteristics as well as biological functions, these HR elicitors from different pathogenic bacteria belong to the harpin protein family. These shared characteristics and their ability to induce HR in a broad range of plants distinguish the harpin protein family from other host specific proteinaceous HR elicitors, for example, elicitins from Phytophthora spp. (Bonnet et al., “Acquired Resistance Triggered by Elicitors in Tobacco and Other Plants,” Eur. J. Plant Path. 102:181-92 (1996); Keller et al. “Physiological and Molecular Characteristics of Elicitin-induced Systemic Acquired Resistance in Tobacco,” Plant Physiol. 110:365-76 (1996)) and avirulence proteins (such as Avr9) from Cladosporium fulvum, which are only able to elicit the HR in a specific variety or species of a plant.

In nature, when certain bacterial infections occur, harpin protein is expressed and then secreted by the bacteria, signaling the plant to mount a defense against the infection. Harpin serves as a signal to activate plant defense and other physiological systems, which include SAR, growth enhancement, and resistance to certain insect damage.

To date, harpin production and use in agricultural and horticultural applications has been as a powdered solid coated on starch. This limits the use and versatility of the harpin proteins, because liquid suspensions of the powdered harpin proteins in water have an effective useful life of only 48-72 hours before significant degradation and loss of activity occurs.

The present invention is directed to overcoming these and other limitations in the art.

### SUMMARY OF THE INVENTION

One aspect of the present invention is directed to a method of making a stable liquid composition containing a harpin protein or polypeptide. This method involves obtaining a liquid extract that is substantially free of cellular debris and comprises a harpin protein or polypeptide. A biocidal agent and, optionally, one or both of a protease inhibitor and a non-ionic surfactant are introduced into the liquid extract, thereby obtaining a liquid composition comprising the harpin protein or polypeptide that retains harpin activity for at least about 72 hours.

Another aspect of the present invention is directed to a composition comprising an aqueous carrier, a harpin protein or polypeptide, an effective amount of a biocidal agent, and optionally, an effective amount of one or both of a protease inhibitor and a non-ionic surfactant. The composition retains harpin activity for at least about 72 hours.

A further aspect of the present invention is directed to a method of inducing a plant response. This method involves applying to a plant or plant seed the composition of the present invention. Application of the composition of the present invention to the plant or plant seed is carried out under conditions effective to induce a plant response.

The present invention is directed to a new method of manufacturing a stable liquid preparation of harpin proteins or polypeptides. As demonstrated in the accompanying Example, stable formulations have been prepared that are capable of retaining significant hypersensitive response inducing-activity over periods of several months. The ability to extend the shelf-life of liquid harpin formulations is of significant importance in the manufacture and distribution of harpin-containing products, because extensive processing to produce powdered harpin-containing formulations are no longer required. This results in several advantages or benefits, including (i) cost savings through the elimination of a powder carrier material and drying processes currently employed in the production of powdered formulations; (ii) avoidance of dust hazard for users; (iii) the liquid formulation is easier to use, because it easily can be diluted in water and mixed with other liquids (other agricultural chemicals to be applied) whereas dissolution of the powder formulations must be monitored; (iv) the liquid formulation can be dispensed more accurately, because it is easier to dispense a proper volume than it is to weigh the correct amount of a powder formulation; and (v) the liquid formulation has the potential to be used as a technical grade material for formulation with other agricultural chemicals.

### DETAILED DESCRIPTION OF THE INVENTION

One aspect of the present invention is directed to a method of making a stable liquid composition containing a harpin protein or polypeptide. This method involves obtaining a liquid extract that is substantially free of cellular debris and comprises a harpin protein or polypeptide. A biocidal agent and, optionally, one or both of a protease inhibitor and a non-ionic surfactant are introduced into the liquid extract, thereby obtaining a liquid composition comprising the harpin protein or polypeptide that retains harpin activity for at least about 72 hours.

As used herein, the term “harpin protein or polypeptide” refers to any member of the art-recognized class of proteins that are produced by plant bacteria, and which share structural features and a capacity for inducing a plant hypersensitive response. Biochemically, these proteins or polypeptides have a number of common structural characteristics. These include being glycine rich, heat stable, hydrophilic, lacking an N-terminal signal sequence, and susceptible to proteolysis. See Bonas, “Bacterial Home Goal by Harpins,” Trends Microbiol. 2:1-2 (1994); Gopalan et al., “Bacterial Genes Involved in the Elicitation of Hypersensitive Response and Pathogenesis,” Plant Disease 80:604-10 (1996); and Alfano et al., “The Type III (Hrp) Secretion Pathway of Plant Pathogenic Bacteria: Trafficking Harpins, Avr Proteins, and Death,” Journal of Bacteriology 179:5655-5662 (1997), each of which is hereby incorporated by reference in its entirety. In addition, harpins share a unique secondary structure that has been associated with their distinct biological activities. The structure has two primary components, an alpha helix unit and a relaxed acidic unit having a sheet or random turn structure. In the absence of one or both of these components, hypersensitive response elicitation does not occur. See PCT Publ. No. WO 01/98501 to Fan et al., which is hereby incorporated by reference in its entirety.

The harpin proteins also share the ability to induce specific plant responses (i.e., following treatment of the plant or a plant seed from which the plant is grown). The induction of plant disease resistance, plant growth, insect resistance, desiccation resistance, and post-harvest disease resistance (in harvested plant products, such as fruits and vegetables) are several of the more important utilities. These uses of the harpin proteins are described in U.S. Pat. No. 6,277,814 to Qiu et al.; U.S. Pat. No. 5,776,889 to Wei et al.; U.S. Pat. No. 5,977,060 to Zitter et al.; U.S. Pat. No. 6,235,974 to Qiu et al.; U.S. Patent Application Publication No. 2003/0104979 to Wei et al.; U.S. Patent Application Publication No. 2002/0019337 to Wei et al.; and U.S. Patent Application Publication No. 2004/0265442; each of which is hereby incorporated by reference in its entirety. The induction of these responses is due to upregulation of jasmonic acid/ethylene and salicylic acid defense pathways, as well as plant growth pathways that regulate photosynthesis and nutrient uptake.

One group of harpin proteins or polypeptides includes, without limitation, homologs of Erwinia amylovora HrpN, which include those from species of Erwinia, Pantoea, and Pectobacterium. Examples of such homologs include those harpin proteins identified at Genbank Accession Nos. AAC31644 (Erwinia amylovora); AAQ21220, AAQ17045, CAE25423, CAE25424, CAE25425, and CAF74881 (Erwinia pyrifoliae); CAC20124, Q47278, Q47279, and AAY17519 (Erwinia chrysanthemi); CAE25422 (Erwinia strain JP557); AAG01466 (Pantoea stewarti); AAF76342 (Pantoea agglomerans); ABZ05760, ABI15988, ABI15989, ABI15990, ABI15991, ABI15992, ABI15996, ABK80762, ABD04037, ABI15994, ABD04035, ABD04036, AAY17521, AAX38231, ABI15995, AAQ73910, and CAL69276 (Pectobacterium carotovorum); YP_{—}050198, AAS20361, and CAE45180 (Pectobacterium atrosepticum); and ABD22989 (Pectobacterium betavasculorum); each of which is hereby incorporated by reference in its entirety.

Another group of harpin proteins or polypeptides includes, without limitation, homologs of Erwinia amylovora HrpW and Pseudomonas syringae HrpW, which includes those from species of Erwinia, Pseudomonas, Xanthomonas, Acidovorax, and Pectobacterium. Examples of such homologs include those harpin proteins identified at Genbank Accession Nos. U94513, CAA74158, AAC04849, and AAF63402 (Erwinia amylovora); AAQ17046 (Erwinia pyrifoliae); YP_{—}001906489 (Erwinia tasmaniensis); YP_{—}050207 (Pectobacterium atrosepticum); AF037983 (Pseudomonas syringae pv. tomato); AA050075 (Pseudomonas syringae pv. phaseolicola); AAL84244 (Pseudomonas syringae pv. maculicola); AAX58537, AAX58557, AAX58525, AAX58531, AAX58527, AAX58577, AAX58491, AAX58515, AAX58517, AAX58523, AAX58583, AAX58451, AAX58561, AAX58453, AAX58541, AAX58589, AAT96311, AAX58497, AAX58579, AAX58449, AAX58485, AAX58563, AAX58581, AAX58575, AAX58569, AAX58567, AAX58505, AAX58591, AAX58503, AAX58507, AAX58509, AAX58469, AAX58441, AAX58543, AAX58495, AAX58549, AAX58593, AAX58511, AAX58519, AAT96270, AAX58447, AAX58571, AAX58465, AAX58489, AAX58533, AAX58535, AAX58461, AAT96350, AAX58473, AAX58483, AAX58475, AAX58457, AAX52461, AAX52457, AAT96222, (Pseudomonas viridiflava); ABA47299 and BAG24117 (Pseudomonas cichorii); CAH57075 (Pseudomonas avellanae); BAE80274 and BAE80242 (Acidovorax avenae); and AAM37767 (Xanthomonas axonopodis pv. citri); each of which is hereby incorporated by reference in its entirety.

Yet another group of harpin proteins or polypeptides includes, without limitation, homologs of Pseudomonas syringae HrpZ, which includes those from other species of Pseudomonas. Examples of such homologs include those harpin proteins identified at Genbank Accession Nos. P35674, AAB00127, ABL01505, AAQ92359, BAD20880, BAD20876, BAD20892, BAD20884, BAD20928, BAD20936, BAD20932, BAD20924, BAD20856, BAD20864, BAD20860, BAD20848, BAD20844, BAD20836, BAD20840, BAD20824, BAD20842, BAD20820, BAD20916, BAD20872, BAC81526, 087653, BAA74798, BAD20904, AAB86735, BAD20912, BAD20908, ABL01504, BAB40655, ABO26225, ABO26228 (Pseudomonas syringae pv.); BAD20868 (Pseudomonas ficuserectae); AAX52452, AAT96159, AAX52266, AAX52396, AAT96322, AAT96281, AAX52272, AAX52306, AAX52270, AAX52402, AAX52276, AAX52318, AAX52262, and AAT96361 (Pseudomonas viridiflava); CAJ76697 (Pseudomonas avellanae); YP_001185537 (Pseudomonas mendocina); and ABA47309 and BAG24128 (Pseudomonas cichorii); each of which is hereby incorporated by reference in its entirety.

An additional group of harpin proteins or polypeptides includes, without limitation, homologs of Xanthomonas campestris HreX (see U.S. Pat. No. 6,960,705 to Wei et al., which is hereby incorporated by reference in its entirety), which includes those from other species of Xanthomonas. Examples of such homologs include those harpin proteins identified at Genbank Accession Nos. NP 636614, YP 001904470, YP_{—}362171 (Xanthomonas campestris); ABB72197, ABK51585, ABU48601, ABK51584, YP_198734, and ZP_{—}02245223 (Xanthomonas oryzae); and ABK51588 and NP_{—}640771 (Xanthomonas axonopodis); each of which is hereby incorporated by reference in its entirety.

Also encompassed by the present invention are stable liquid formulations that contain hypersensitive response eliciting fragments of the above-listed harpin protein or polypeptides. Preferred fragments include two structural units: a stable α-helix unit with 12 or more amino acids in length; and a hydrophilic, acidic unit with 12 or more amino acids in length, which could be a beta-form, a beta-turn, or unordered forms. Preferred fragments also are characterized by an acidic pI value, that is preferably below 5. Preferred fragments contain between about 28 to about 40 amino acids, although fewer or greater amino acid residues can be present.

Examples of suitable fragments are identified in U.S. Pat. No. 6,583,107 to Laby et al., and PCT Publication No. WO 01/098501 to Fan et al., each of which is hereby incorporated by reference in its entirety. PCT Publication No. WO 01/098501 to Fan et al. also describes methods for obtaining fragments of harpin protein or polypeptides that could be employed in the present invention.

Suitable HR-eliciting polypeptide fragments include, without limitation, those identified in Table 1 below:

TABLE 1
List of HR-Eliciting Fragments

| HR domain | Isolated Source | Amino Acid Residues | pI |
|---|---|---|---|
| HrpN_{Ea}-1 | E. amylovora | 43-70 | 3.09 |
| HrpN_{Ea}-2 | E. amylovora | 140-176 | 3.17 |
| HrpN_{Ech}-1 | E. chrysanthemi | 78-118 | 5.25 |
| HrpN_{Ech}-2 | E. chrysanthemi | 256-295 | 4.62 |
| HrpN_{Ecc}-1 | E. carotovora | 25-63 | 4.06 |
| HrpN_{Ecc}-2 | E. carotovora | 101-140 | 3.00 |
| HrpW_{Pss}-1 | P. syringae | 52-96 | 4.32 |
| HrpW_{Ea}-1 | E. amylovora | 10-59 | 4.53 |
| HrpZ_{Pss}-1 | P. syringae | 97-132 | 3.68 |
| HrpZ_{Pss}-2 | P. syringae | 153-189 | 3.67 |
| HrpZ_{Pss}-3 | P. syringae | 271-308 | 3.95 |
| PopA1_{Rs}-1 | R. solanacearum | 92-125 | 3.75 |
| PopA1_{Rs}-2 | R. solanacearum | 206-260 | 3.62 |

Suitable fragments of harpin protein or polypeptides may not elicit the hypersensitive response in plants, but may still be useful in the formulations and compositions of the present invention. Such fragments are described in U.S. Pat. No. 6,858,707 to Wei et al., which is hereby incorporated by reference in its entirey.

Suitable fragments can be produced by several means. According to one approach, subclones of the gene encoding a known harpin protein or polypeptide are produced by conventional molecular genetic manipulation by subcloning gene fragments. The subclones then are expressed in vitro or in vivo in bacterial cells to yield a smaller protein or peptide that can be tested for activity.

As an alternative approach, fragments can be produced by digestion of a full-length harpin protein or polypeptide with proteolytic enzymes like chymotrypsin or Staphylococcus proteinase A, or trypsin. Different proteolytic enzymes are likely to cleave elicitor proteins at different sites based on the amino acid sequence of the harpin protein. Some of the fragments that result from proteolysis may be active elicitors of resistance.

In yet another approach, based on knowledge of the primary structure of the protein, fragments of the harpin protein gene may be synthesized by using the PCR technique together with specific sets of primers chosen to represent particular portions of the protein. These then would be cloned into an appropriate vector for expression of a truncated peptide or protein.

Chemical synthesis can also be used to make suitable fragments. Such a synthesis is carried out using known amino acid sequences for the harpin being produced. Alternatively, subjecting a full length harpin to high temperatures and pressures will produce fragments. These fragments can then be separated by conventional procedures (e.g., chromatography, SDS-PAGE).

Harpin protein or polypeptides of the present invention may also include isolated hypersensitive response elicitor proteins comprising a pair or more of spaced apart HR-eliciting domains, each comprising an acidic portion linked to an alpha-helix and capable of eliciting a hypersensitive response in plants, as described in PCT Publication No. WO 01/098501 to Fan et al., which is hereby incorporated by reference in its entirety. For example, building blocks containing one or more HR-eliciting domains include, without limitation, the building blocks identified in Table 2 below:

TABLE 2
Superharpin Building Block Domains

| Domain | | | | |
|---|---|---|---|---|
| Sequence | Source | MW (kDa) | #a.a. | pI |
| A | PopA70-146 | 10.69 | 104 | 6.48 |
| (N_{N}) | HrpN_{Ea}40-80 | 6.754 | 68 | 6.78 |
| (N_{N})₂ | Dimer of HrpN_{Ea}40-80 | 10.84 | 111 | 6.13 |
| (N_{N})₃ | Triplemer of HrpN_{Ea}40-80 | 14.93 | 154 | 5.63 |
| (N_{N})₄ | Tetramer of HrpN_{Ea}40-80 | 19.01 | 197 | 4.95 |
| (N_{C}) | HrpN_{Ea}140-180 | 7.224 | 68 | 5.01 |
| (N_{C})₂ | Dimer of HrpN_{Ea}140-180 | 11.78 | 111 | 3.98 |
| (N_{C})₃ | Trimer of HrpN_{Ea}140-180 | 16.34 | 154 | 3.72 |
| (N_{C})₄ | Tetramer of HrpN_{Ea}140-180 | 20.89 | 197 | 3.58 |
| (N_{C})₁₀ | Decamer of HrpN_{Ea}140-180 | 48.23 | 455 | 3.28 |
| (N_{C})₁₆ | Hexadecamer of HrpN_{Ea}140-180 | 75.57 | 713 | 3.18 |
| W | HrpW_{Ea}10-59 | 7.986 | 77 | 6.48 |
| Z_{N} | HrpZ90-150 | 8.087 | 78 | 5.38 |
| Z₂₆₆₋₃₀₈ | HrpZ266-308 | 7.029 | 70 | 6.40 |

With the combination of these (and other) HR-eliciting domains, new harpin polypeptides (i. e., superharpins) can be produced that have higher HR potency and have enhanced ability to induce desired plant response (e.g., disease resistance, insect resistance, enhanced growth, environmental stress tolerance, and post-harvest disease resistance). Superharpins can be formed using one HR domain repeat unit (cancatomer), different combinations of HR domains, and/or biologically active domains from other elicitors.

Using these building blocks, several isolated superharpin proteins include, without limitation, those identified in Table 3 below:

TABLE 3
Superharpin Constructions

| Protein | Domain Sequence | MW (kDa) | # a.a. | pI |
|---|---|---|---|---|
| SH-1 | *W(N_{N})₄A(N_{C})₄Z₂₆₆₋₃₀₈ | 54.955 | 545 | 3.69 |
| SH-2 | *W(N_{N})₄Z_{N}(N_{C})₄Z₂₆₆₋₃₀₈ | 52.341 | 519 | 3.54 |
| SH-3 | *W(N_{N})₄Z_{N}(N_{C})₄Z₂₆₆₋₃₀₈A | 60.375 | 598 | 3.67 |

These superharpins are heat stable and soluble, and have been demonstrated to possess improved growth enhancement and/or disease resistance activity as compared to harpin proteins isolated from plant pathogenic bacteria, such as HrpN. These superharpins are described in PCT Publication No. WO 01/098501 to Fan et al., which is hereby incorporated by reference in its entirety.

One preferred superharpin protein, now commercially available from Plant Healthcare Inc., is characterized by the amino acid sequence of SEQ ID NO: 1 as follows:

MSLNTSGLGASTMQISIGGAGGNNGLLGTHMPGTSSSPGLFQSGGDNGLG
GHNANSALGQQPIDRQTIEQMAQLLAELLKSLLDSGEKLGDNFGASADSA
SGTGQQDLMTQVLNGLAKSMLDDLLTKQDGGTSFSEDDSGPAKDGNANAG
ANDPSKNDPSKSQGPQSANKTGNVDDANNQDPMQALMQLLEDLVKLLKAA
LHMQQPGGNDKGNGVGGDSGQNDDSTSGTDSTSDSSDPMQQLLKMFSEIM
QSLFGDEQDGTDSTSGSRFTRTGIGMKAGIQALNDIGTHSDSSTRSFVNK
GDRAMAKEIGQFMDQYPEVFGKPQYQKGPGQEVKTDDKSWAKALSKPDDD
GMTPASMEQFNKAKGMIKSAMAGDTGNGNLQARGAGGSSLGIDAMMAGDA
INNMALGKLGAA

Residues 1-30 correspond to the N-terminal sequence of HrpN_{Ea}; residues 31-34 (bold) are artifacts of ligating the HR domains together; residues 35-83 correspond to one HR domain of HrpW_{Ea} (residues 10-59); residues 84-86 (bold) are artifacts of ligating the HR domains together; residues 87-138 correspond to one HR domain of HrpZ_{Pss} (residues 90-141); residues 139-140 (bold) are artifacts of ligating the HR domains together; residues 141-211 correspond to one HR domain of PopA (residues 70-140); residues 212-220 correspond to artifacts of ligating the HR domains together; residues 221-261 correspond to one HR domain of HrpN_{Ea} (residues 140-180); residues 262-271 correspond to artifacts of ligating the HR domains together; and residues 272-412 correspond to the C-terminal sequence of HrpN_{Ea} (residues 263-403).

The superharpin protein of SEQ ID NO: 1 is encoded by the nucleotide sequence of SEQ ID NO: 2 as follows:

ATGAGTCTGAATACAAGTGGGCTGGGAGCGTCAACGATGCAAATTTCTAT
CGGCGGTGCGGGCGGAAATAACGGGTTGCTGGGTACGCATATGCCCGGGA
CCTCGTCCTCGCCGGGTCTGTTCCAGTCCGGGGGGGACAACGGGCTTGGT
GGTCATAATGCAAATTCTGCGTTGGGGCAACAACCCATCGATCGGCAAAC
CATTGAGCAAATGGCTCAATTATTGGCGGAACTGTTAAAGTCACTGCTAG
ATAGTGGGGAAAAGCTCGGTGACAACTTCGGCGCGTCTGCGGACAGCGCC
TCGGGTACCGGACAGCAGGACCTGATGACTCAGGTGCTCAATGGCCTGGC
CAAGTCGATGCTCGATGATCTTCTGACCAAGCAGGATGGCGGGACCAGCT
TCTCCGAAGACGATAGTGGGCCGGCGAAGGACGGCAATGCCAACGCGGGC
GCCAACGACCCGAGCAAGAACGACCCGAGCAAGAGCCAGGGTCCGCAGTC
GGCCAACAAGACCGGCAACGTCGACGACGCCAACAACCAGGATCCGATGC
AAGCGCTGATGCAGCTGCTGGAAGACCTGGTGAAGCTGCTGAAGGCGGCC
CTGCACATGCAGCAGCCCGGCGGCAATGACAAGGGCAACGGCGTGGGCGG
TGATAGTGGGCAAAACGACGATTCCACCTCCGGCACAGATTCCACCTCAG
ACTCCAGCGACCCGATGCAGCAGCTGCTGAAGATGTTCAGCGAGATAATG
CAAAGCCTGTTTGGTGATGAGCAAGATGGCACCGATAGTACTAGCGGCTC
GAGGTTTACTCGTACCGGTATCGGTATGAAAGCGGGCATTCAGGCGCTGA
ATGATATCGGTACGCACAGCGACAGTTCAACCCGTTCTTTCGTCAATAAA
GGCGATCGGGCGATGGCGAAGGAAATCGGTCAGTTCATGGACCAGTATCC
TGAGGTGTTTGGCAAGCCGCAGTACCAGAAAGGCCCGGGTCAGGAGGTGA
AAACCGATGACAAATCATGGGCAAAAGCACTGAGCAAGCCAGATGACGAC
GGAATGACACCAGCCAGTATGGAGCAGTTCAACAAAGCCAAGGGCATGAT
CAAAAGCGCCATGGCGGGTGATACCGGCAACGGCAACCTGCAGGCACGCG
GTGCCGGTGGTTCTTCGCTGGGTATTGATGCCATGATGGCCGGTGATGCC
ATTAACAATATGGCACTTGGCAAGCTGGGCGCGGCTTAA

According to the present invention, the method of making a stable liquid composition containing a harpin protein or polypeptide involves obtaining a liquid extract that is substantially free of cellular debris and comprises a harpin protein or polypeptide. This can be carried out by fermenting a suspension of harpin protein or polypeptide-producing plant bacteria. Harpin protein or polypeptides can be produced readily through fermentation in rapidly growing bacteria. For example, recombinant Escherichia coli may be used for large-scale harpin protein or polypeptide production. Current technology enables the production of relatively large intracellular concentrations of harpin proteins or polypeptides.

Recombinant methodoligies generally involve inserting a DNA molecule expressing a protein or polypeptide of interest into an expression system to which the DNA molecule is heterologous (i. e., not normally present). The heterologous DNA molecule is inserted into the expression system or vector in proper sense orientation and correct reading frame. The vector contains the necessary elements for the transcription and translation of the inserted protein-coding sequences. Transcription of DNA is dependent upon the presence of a promoter. Similarly, translation of mRNA in prokaryotes depends upon the presence of the proper prokaryotic signals which differ from those of eukaryotes. For a review on maximizing gene expression, see Roberts and Lauer, Methods in Enzymology 68:473 (1979), which is hereby incorporated by reference.

Regardless of the specific regulatory sequences employed, the DNA molecule is cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety. Once the isolated DNA molecule encoding the harpin protein or polypeptide has been cloned into an expression system, it is ready to be incorporated into a host cell. Such incorporation can be carried out by the various forms of transformation, depending upon the vector/host cell system. Suitable host cells include, but are not limited to, bacteria, virus, yeast, mammalian cells, insect, plant, and the like.

Optionally, the recombinant host cells can be host cells that express a native or recombinant, functional type III secretion system. This is described in detail in U.S. Pat. No. 6,596,509 to Bauer et al., which is hereby incorporated by reference in its entirety. As a consequence of expressing the functional type III secretion system, the cells will express the harpin protein or polypeptide and then secrete the protein into the culture medium. This can simplify isolation and purification of the harpin protein or polypeptide.

The recombinant host cells can be grown in appropriate fermentation chambers, preferably under temperature and nutrient conditions that optimize growth of the host cells and the expression of the harpin proteins or polypeptides. Persons of skill in the art are fully able to identify optimal conditions for particular host cells.

After fermentation, the bacterial suspension may be diluted in, e.g., about 2 to 5 fold volume of a buffer to adjust the pH between about 5.5 to 10, more preferably to a pH of between about 7 to 9, and even more preferably to a pH of about 8.0. Suitable buffers are well-known in the art and may include, for example, potassium phosphate buffer or a Tris-EDTA buffer. The concentration of the buffer can be from about 0.001 mM to about 0.5 M.

Following the pH adjustment, the bacterial suspension solution is heat treated to a temperature of about 60-130° C., preferably to a temperature of about 95-125° C. Heat treatment may be carried out for any suitable period of time. In one embodiment, heat treatment is carried out for a period of about five minutes up to about 30 minutes.

The heated suspension solution is then cooled. A suitable cool down temperature is, without limitation, about 35-55° C., preferably about 45° C.

Following cooling, bacterial cells in the bacterial suspension are lysed, if required, to liberate the harpin protein or polypeptide. Cell lysis may be carried out, e.g., by contacting the bacterial suspension with a lysozyme. The concentration of lysozyme may be anywhere from about 2 ppm to 100 ppm. Alternatively, cell lysis may involve non-chemical methods, such as high pressure or sonication, both of which are well known by persons of ordinary skill in the art.

It may be desirable, after cell lysis, to incubate the bacterial suspension. Suitable incubation times may vary. For example, it may be desirable to incubate the bacterial suspension for a period of about 30-45 minutes at a temperature of about 40-42° C.

After lysing, the desired protein or polypeptide (i.e., harpin protein or polypeptide) can be further extracted by removing the cell debris and the denatured proteins resulting from the previous heat treatment step. In one embodiment, the extract is centrifuged for about 10-20 minutes to remove some of the cell debris. Suitable centrifuge speeds may be from about 4,000 to 20,000 rpm and the spinning down time can be from about 10 minutes to 20 minutes. Further cell debris may then be removed by heat treating and centrifuging the supernatant to obtain a liquid extract that is substantially free of cellular debris by removing more than about 60%, 70%, 80%, 90%, or 95% of total solids. This subsequent heat treatment may be carried out at a temperature of about 60° C. for up to about two hours, at about 100° C. for about 10 minutes, or at about 121° C. with 15 psi of pressure for about 5 minutes. These temperatures and times may vary depending on other conditions.

The method of making a stable liquid composition containing a harpin protein or polypeptide of the present invention further involves introducing into the liquid extract a biocidal agent and, optionally, one or both of a protease inhibitor and a non-ionic surfactant, thereby obtaining a liquid composition comprising the harpin protein or polypeptide. In one embodiment, a protease inhibitor is introduced into the liquid extract without a non-ionic surfactant. In another embodiment, a non-ionic surfactant is introduced into the liquid extract without a protease inhibitor. In a further embodiment, both a protease inhibitor and a non-ionic surfactant are introduced into the liquid extract. In yet another embodiment, neither a protease inhibitor nor a non-ionic surfactant are introduced into the liquid extract.

Biocidal agents are added to the liquid extract for preservation. Suitable biocidal agents include, without limitation, antibiotics, toxic chemicals, and disinfectants. For example, a suitable antibiotic is streptomycin, a suitable toxic agent is sodium azide, and a suitable disinfectant is a Triple Action disinfectant (i. e., the EPA approved pesticide with the following active ingredients: 1-decanaminium, N,N-dimethyl-N-octyl-, chloride (12.4% by mass); 1-octanaminium, N,N-dimethyl-N-octyl-, chloride (12.4% by mass); alkyl(C12-16)dimethylbenzylammonium chloride (12.4% by mass); sodium carbonate (3% by mass); and edentate sodium (2.5% by mass)). The concentration of biocidal agent introduced may be in the range of about 1 ppm to about 100 ppm, more preferably about 2 ppm to about 30 ppm, most preferably about 5 ppm to about 10 ppm.

Protease inhibitors may be added to prevent harpin degradation by residual proteases in the harpin extract. Protease inhibitors include various inhibitors classed by protease type or by their mechanism of action. Suitable protease inhibitors may include, without limitation, cysteine protease inhibitors, serine protease inhibitors (serpins), trypsin inhibitors, threonine protease inhibitors, aspartic acid protease inhibitors, and metalloprotease inhibitors. Suitable protease inhibitors may be selected according to their mechanism of action. For example, suitable protease inhibitors may include, without limitation, suicide inhibitors, transition state inhibitors, protein protease inhibitors, and chelating agents. Examples of commercially available protease inhibitors include, without limitation, aprotinin, bestatin, calpain inhibitor I, calpain inhibitor II, chymostatin, E-64, leupeptin (N-acetyl-L-leucyl-L-leucyl-L-argininal), alpha-2-macroglobuline, pefabloc SC, pepstatin, PMSF (phenylmethanesulfonyl fluoride), and tosyl-L-lysine chloromethyl ketone (TLCK).

Protease inhibitors may be added to the extract at a concentration of about 1 ppm to about 100 ppm, more preferably about 2 ppm to about 30 ppm, most preferably about 5 ppm to about 10 ppm.

Suitable non-ionic surfactants include, without limitation, sorbitan fatty acid ester, glycerin fatty acid ester, fatty acid polyglyceride, fatty acid alcohol polyglycol ether, acetylene glycol, acetylene alcohol, oxyalkylene block polymer, polyoxyethylene alkyl ether, polyoxyethylene alkylaryl ether, polyoxyethylene styrylaryl ether, polyoxyethylene glycol alkyl ether, polyoxyethylene fatty acid ester, polyoxyethylene sorbitan fatty acid ester, polyoxyethylene glycerin fatty acid ester, polyoxyethylene hydrogenated castor oil, and polyoxypropylene fatty acid ester.

Non-ionic surfacatants may be added to the extract at a volume amount of about 0.005 to about 20%, more preferably about 0.01 to about 15%, most preferably about 0.05% to about 10%.

As a result of introducing the biocidal agent and, optionally, the protease inhibitor and surfactant as described above, the compositions of the present invention are characterized by maintaining their harpin activity for at least 72 hours and preferably much longer. Preferably, the liquid composition produced by the methods of the present invention retains harpin activity for more than about 5 days, 1 week, 2 weeks, 3 weeks, or 4 weeks, more preferably at least about 2 to 3 months, and most preferably longer than about 4 to 6 months. As used herein, retention of harpin activity can be determined by comparing the activity of the aged liquid composition to a recently prepared liquid composition or to a prior assessment made on the same composition. The activity can be measured by the effects of the composition on plants as assessed by the disease resistance, growth enhancement, stress resistance, etc., of the plants following challenge. Preferably, the compositions of the present invention retain (for more than 72 hours) at least about 70% activity, more preferably at least about 70% to about 80% activity, and most preferably at least about 80% to 90% activity.

Alternatively, the stability of the liquid composition of the present invention can be assessed using, e.g., HPLC analysis or other suitable procedures that can identify quantity of a specific protein or polypeptide. The stability of harpin protein or polypeptide in a composition of the present invention can be determined by comparing the quantity of harpin protein in the aged liquid composition to that of a recently prepared liquid composition or to a prior quantitation performed on the same composition. The measurement of harpin protein stability strongly correlates with retention of activity.

Another aspect of the present invention is directed to a composition comprising an aqueous carrier, a harpin protein or polypeptide, an effective amount of a biocidal agent, and optionally, an effective amount of one or both of a protease inhibitor and a non-ionic surfactant. The composition retains harpin activity for at least about 72 hours.

The composition of the present invention may be formulated into any suitable form including, without limitation, a solution, emulsion, emulsifiable concentrate, suspension, foam, paste, aerosol, suspoemulsion concentrate, or slurry. Suitable compositions include those for HV, LV, and ULV spraying and for ULV cool and warm fogging formulations. Preferably, the composition of the present invention is formulated in a manner suitable for large or small scale agricultural and horticultural applications.

These formulations are produced in a known manner, for example, by mixing the liquid composition with extenders, that is, liquid solvents, liquefied gases under pressure, and/or solid carriers. Wetting agents and/or surfactants, that is, emulsifiers and/or dispersants, sequestering agents, plasticizers, brighteners, flow agents, coalescing agents, waxes, fillers, polymers, anti-freezing agents, biocides, thickeners, tackifiers, and/or foam formers and defoaming agents may also be used in manners commonly known by those of ordinary skill in the art. If the extender used is water, it is also possible to employ, for example, organic solvents as auxiliary solvents. Other possible additives are mineral and vegetable oils, colorants such as inorganic pigments, and trace nutrients.

The nature and action of such additives are well-known to those of ordinary skill in the art of liquid formulations. Additives should not interfere with the action of the harpin proteins or polypeptides or any other biologically active component included in the formulation.

The active compound content of the harpin protein or polypeptide contained in the formulation of the present invention may vary within a wide range. For example, the concentration of active compound (i.e., active harpin protein or polypeptide) may be from 0.0000001 to 20% by weight, and is preferably from 0.00001 to 15% by weight.

In one embodiment, it may be desirable to combine the composition of the present invention with effective amounts of other agricultural or horticultural chemicals, such as herbicides (e.g., glyphosate), insecticides, acaracides, nematicides, molluscicides, attractants, sterilants, bactericides, acaricides, nematicides, fungicides, and/or growth regulators.

One preferred herbicide is glyphosate, commonly known as 2 (phosphonomethylamino)acetic acid. Glyphosate salts may also be used. Suitable glyphosate salts include, for example, but are not limited to, isopropylamine salts, diammonium salts, and trimethylsulfonium salts. Mixtures including glyphosate typically include one or more surfactants, typically one or more nonionic surfactants, though no surfactant should be required. Glyphosate-containing formulations are typically applied to desirable plants and plant-parts that are glyphosate resistant.

Examples of other herbicides useful in compositions described herein include, for example, but are not limited to: amide herbicides, including allidochlor, amicarbazone, beflubutamid, benzadox, benzipram, bromobutide, cafenstrole, CDEA, cyprazole, dimethenamid, dimethenamid-P, diphenamid, epronaz, etnipromid, fentrazamide, flucarbazone, flupoxam, fomesafen, halosafen, isocarbamid, isoxaben, napropamide, naptalam, pethoxamid, propyzamide, quinonamid, saflufenacil, and tebutam; anilide herbicides, including chloranocryl, cisanilide, clomeprop, cypromid, diflufenican, etobenzanid, fenasulam, flufenacet, flufenican, ipfencarbazone, mefenacet mefluidide, metamifop, monalide, naproanilide, pentanochlor, picolinafen, propanil, sulfentrazone; arylalanine herbicides, including benzoylprop, flamprop, and flamprop-M; chloroacetanilide herbicides, including acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor, and xylachlor; sulfonanilide herbicides, including benzofluor, cloransulam, diclosulam, florasulam, flumetsulam, metosulam, perfluidone, pyrimisulfan, and profluazol; sulfonamide herbicides, including asulam, carbasulam, fenasulam, oryzalin, penoxsulam, and pyroxsulam; thioamide herbicides, including bencarbazone and chlorthiamid; antibiotic herbicides, including bilanafos; aromatic acid herbicides; benzoic acid herbicides, including chloramben, dicamba, 2,3,6-TBA, and tricamba; pyrimidinyloxybenzoic acid herbicides, including bispyribac, and pyriminobac; pyrimidinylthiobenzoic acid herbicides, including pyrithiobac; phthalic acid herbicides, including chlorthal, picolinic acid herbicides, aminopyralid, clopyralid, and picloram; quinolinecarboxylic acid herbicides, including quinclorac, and quinmerac; arsenical herbicides, including cacodylic acid, CMA, DSMA, hexaflurate, MAA, MAMA, MSMA, potassium arsenite, and sodium arsenite; benzoylcyclohexanedione herbicides, including mesotrione, sulcotrione, tefuryltrione, and tembotrione; benzofuranyl alkylsulfonate herbicides, including benfuresate, and ethofumesate; benzothiazole herbicides, including benazolin, benzthiazuron, fenthiaprop, mefenacet, and methabenzthiazuron; carbamate herbicides, including asulam, carboxazole, chlorprocarb, dichlormate, fenasulam, karbutilate, and terbucarb; carbanilate herbicides, including barban, BCPC, carbasulam, carbetamide, CEPC, chlorbufam, chlorpropham, CPPC, desmedipham, phenisopham, phenmedipham, phenmedipham-ethyl, propham, and swep; cyclohexene oxime herbicides, including alloxydim, butroxydim, clethodim, cloproxydim, cycloxydim, profoxydim, sethoxydim, tepraloxydim, and tralkoxydim; cyclopropylisoxazole herbicides, including isoxachlortole and isoxaflutole; dicarboximide herbicides, including cinidon-ethyl, flumezin, flumiclorac, flumioxazin, and flumipropyn; dinitroaniline herbicides, including benfluralin, butralin, dinitramine, ethalfluralin, fluchloralin, isopropalin, methalpropalin, nitralin, oryzalin, pendimethalin, prodiamine, profluralin, and trifluralin; dinitrophenol herbicides, including dinofenate, dinoprop, dinosam, dinoseb, dinoterb, DNOC, etinofen, and medinoterb; diphenyl ether herbicides, including ethoxyfen; nitrophenyl ether herbicides, including acifluorfen, aclonifen, bifenox, chlomethoxyfen, chlornitrofen, etnipromid, fluorodifen, fluoroglycofen, fluoronitrofen, fomesafen, furyloxyfen, halosafen, lactofen, nitrofen, nitrofluorfen, and oxyfluorfen; dithiocarbamate herbicides, including dazomet and metam; halogenated aliphatic herbicides, including alorac, chloropon, dalapon, flupropanate, hexachloroacetone, iodomethane, methyl bromide, monochloroacetic acid, SMA, and TCA; imidazolinone herbicides, including imazamethabenz, imazamox, imazapic, imazapyr, imazaquin, and imazethapyr; inorganic herbicides, including ammonium sulfamate, borax, calcium chlorate, copper sulfate ferrous sulfate, potassium azide, potassium cyanate, sodium azide, sodium chlorate, and sulfuric acid; nitrile herbicides, including bromobonil, bromoxynil, chloroxynil, dichlobenil, iodobonil, ioxynil, and pyraclonil; organophosphorus herbicides, including amiprofos-methyl, anilofos, bensulide, bilanafos, butamifos, 2,4-DEP, DMPA, EBEP, fosamine, glufosinate, glufosinate-P, glyphosate, and piperophos; oxadiazolone herbicides, including dimefuron, methazole, oxadiargyl, and oxadiazon; oxazole herbicides, including carboxazole, isouron, isoxaben, isoxachlortole, isoxaflutole, monisouron, pyroxasulfone, and topramezone; phenoxy herbicides, including bromofenoxim, clomeprop, 2,4-DEB, 2,4-DEP, difenopenten, disul, erbon, etnipromid, fenteracol, and trifopsime; phenoxyacetic herbicides, including 4-CPA, 2,4-D, 3,4-DA, MCPA, MCPA-thioethyl, and 2,4,5-T; phenoxybutyric herbicides, including 4-CPB, 2,4-DB, 3,4-DB, MCPB, and 2,4,5-TB; phenoxypropionic herbicides, including cloprop, 4-CPP, dichlorprop, dichlorprop-P, 3,4-DP, fenoprop, mecoprop, and mecoprop-P; aryloxyphenoxypropionic herbicides, including chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P, and trifop; phenylenediamine herbicides, including dinitramine, and prodiamine; pyrazole herbicides, including azimsulfuron, difenzoquat, halosulfuron, metazachlor, pyrazosulfuron, and pyroxasulfone; benzoylpyrazole herbicides, including benzofenap, pyrasulfotole, pyrazolynate, pyrazoxyfen, and topramezone; phenylpyrazole herbicides, including fluazolate, nipyraclofen, and pyraflufen; pyridazine herbicides, including credazine, pyridafol, and pyridate; pyridazinone herbicides, including brompyrazon, chloridazon, dimidazon, flufenpyr, metflurazon, norflurazon, oxapyrazon, and pydanon; pyridine herbicides, including aminopyralid, cliodinate, clopyralid, diflufenican, dithiopyr, flufenican, fluroxypyr, haloxydine, picloram, picolinafen, pyriclor, pyroxsulam, thiazopyr, and triclopyr; pyrimidinediamine herbicides, including iprymidam and tioclorim; quaternary ammonium herbicides, including cyperquat, diethamquat, difenzoquat, diquat, morfamquat, and paraquat; thiocarbamate herbicides, including butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate, and vernolate; thiocarbonate herbicides, including dimexano, EXD, and proxan; thiourea herbicides, including methiuron; triazine herbicides, including dipropetryn, triaziflam, and trihydroxytriazine; chlorotriazine herbicides, including atrazine, chlorazine, cyanazine, cyprazine, eglinazine, ipazine, mesoprazine, procyazine, proglinazine, propazine, sebuthylazine, simazine, terbuthylazine, and trietazine; methoxytriazine herbicides, including atraton, methometon, prometon, secbumeton, simeton, and terbumeton; methylthiotriazine herbicides, includingametryn, aziprotryne, cyanatryn, desmetryn, dimethametryn, methoprotryne, prometryn, simetryn, terbutryn, triazinone herbicides, including ametridione, amibuzin, hexazinone, isomethiozin, metamitron, metribuzin, triazole herbicides, including amitrole, cafenstrole, epronaz, and flupoxam; triazolone herbicides, including amicarbazone, bencarbazone, carfentrazone, flucarbazone, ipfencarbazone, propoxycarbazone, sulfentrazone, and thiencarbazone; triazolopyrimidine herbicides, including cloransulam, diclosulam, florasulam, flumetsulam, metosulam, penoxsulam, pyroxsulam, uracil herbicides, including benzfendizone, bromacil, butafenacil, flupropacil, isocil, lenacil, saflufenacil, and terbacil; urea herbicides, including benzthiazuron, cumyluron, cycluron, dichloralurea, diflufenzopyr, isonoruron, isouron, methabenzthiazuron, monisouron, and noruron; phenylurea herbicides, including anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluron, phenobenzuron, siduron, tetrafluron, and thidiazuron; sulfonylurea herbicides; pyrimidinylsulfonylurea herbicides, including amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron, and trifloxysulfuron; triazinylsulfonylurea herbicides, including chlorsulfuron, cinosulfuron, ethametsulfuron, iodosulfuron, metsulfuron, prosulfuron, thifensulfuron, triasulfuron, tribenuron, triflusulfuron, and tritosulfuron; thiadiazolylurea herbicides, including buthiuron, ethidimuron, tebuthiuron, thiazafluron, and thidiazuron; and unclassified herbicides, including acrolein, allyl alcohol, aminocyclopyrachlor, azafenidin, bentazone, benzobicyclon, buthidazole, calcium cyanamide, cambendichlor, chlorfenac, chlorfenprop, chlorflurazole, chlorflurenol, cinmethylin, clomazone, CPMF, cresol, cyanamide, ortho-dichlorobenzene, dimepiperate, endothal, fluoromidine, fluridone, flurochloridone,flurtamone, fluthiacet, indanofan, methyl isothiocyanate, OCH, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan, and tritac. The above list is exemplary only and other herbicides may be used and would fall within the scope of the present invention.

Examples of specific insecticides, acaracides, nematicides, and molluscicides that may be used in compositions taught herein include, but are not limited to: abamectin; acephate; acetamiprid; acrinathhn; alanycarb; aldicarb; alpha-cypermethrin; alphamethrin; amitraz; azinphos A; azinphos-methyl; azocyclotin; bendiocarb; benfuracarb; bensultap; beta cyfluthrin; bifenthrin; brofenprox; bromophos A; bufencarb; buprofezin; butocarboxin; butylpyridaben; cadusafos; carbaryl; carbofuran; carbophenothion; carbosulfan; cartap; chloethocarb; chloranthraniliprole; chloroethoxyfos; chlorfenvenphos; chlorofluazuron; chloromephos; chloropyrifos; cis-res-methrin; clocythrin; clofentezin; clothianidin; cyanoimine; cyanophos; cycloprothhn; cyfluthrin; cyhexatin; deltamethrin; demeton M; demeton S; demeton-S-methyl; diafenthiuron; dibutylaminothio; dichlofenthion; dicliphos; diethion; diflubenzuron; dimethoate; dimethylvinphos; dinotefuran; dioxathion; doramectin; edifenphos; emamectin; endosulfan; esfenvalerate; ethiofencarb; ethion; ethiprole; ethofenprox; ethoprophos; etrimphos; fenamiphos; fenazaquin; fenbutatin oxide; fenitrothion; fenobucarb; fenothiocarb; fenoxycarb; fenpropathrin; fenpyrad; fenpyroximate; fenthion; fenvalerate; fipronil; fluazinam; flubendiamide; flucycloxuron; flucythrinate; flufenoxuron; flufenprox; fluxofenime; fonophos; formothion; fosthiazate; fubfenprox; gamma cyhalothrin; HCH; heptenophos; hexaflumuron; hexythiazox; imidacloprid; iprobenfos; isoprocarb; isoxathion; ivermectin, lambda cyhalothrin; lindane; lufenuron; malathion; mecarbam; mesulfenphos; metaldehyde; methamidophos; methiocarb; methomyl; metolcarb; mevinphos; milbemectin; milbemycin oxime; moxidectin; naled; NC 184; nitenpyram; nitromethylene; omethoate; oxamyl; oxydemethon M; oxydeprofos; parathion; parathion-methyl; permethrin; phenthoate; phorate; phosalone; phosmet; phoxim; pirimicarb; pirimiphos A; pirimiphos M; promecarb; propaphos; propoxur; prothiofos; prothoate; pymetrozine; pyrachlophos; pyrada-phenthion; pyresmethrin; pyrethrum; pyridaben; pyrimidifen; pyripfoxyfen; pyriproxyfen; rynaxypyr; salithion; sebufos; silafluofen; sulfotep; sulprofos; tebufenozide; tebufenpyrad; tebupihmphos; teflubenzuron; tefluthrin; temephos; terbam; terbufos; tetrachloro-vinphos; thiacloprid; thiafenox; thiamethoxam; thiodicarb; thiofanox; thionazin; thuringiensin; tralomethrin; triarthen; triazamate; triazophos; triazuron; trichlorofon; triflumuron; trimethacarb; vamidothion; xylylcarb; zeta-cypermethrin; zetamethrin; and Bacillus thuringiensis (Bt) products, including the salts and esters thereof. The above list is exemplary only and other insecticides may be used and would fall within the scope of the present invention.

A variety of fungicides may be used in embodiments of the present invention. They include, for example, those classified and listed by the Fungicide Resistance Action Committee (FRAC), FRAC CODE LIST 1: Fungicides sorted by FRAC Code, December 2006, which is hereby incorporated by reference in its entirety. A summary of this list includes: Methyl benzimidazole carbamates (MBC): e.g., benzimidazoles and thiophanates; Dicarboximides; Demethylation inhibitors (DMI) (SBI: Class I): e.g., imidazoles, piperazines, pyridines, pyrimidines, and triazoles; Phenylamides (PA): e.g., acylalanines, oxazolidinones, and butyrolactones; Amines (SBI: Class II): e.g., morpholines, piperidines, and spiroketalamines; Phosphoro-thiolates and Dithiolanes; Carboxamides: e.g., benzamides, furan carboxamides, oxathiin carboxamides, thiazole carboxamides, pyrazole carboxamides, and pyridine carboxamides; Hydroxy-(2-amino-) pyrimidines; Anilino-pyrimidines (AP); N-phenyl carbamates; Quinone outside inhibitors (QoI): e.g., methoxyacrylates, methoxy-carbamates, oximino acetates, oximino-acetamides, oxazolidine-diones, dihydro-dioxazines, imidazolinones, and benzyl-carbamates; Phenylpyrroles; Quinolines; Aromatic hydrocarbons (AH) and Heteroaromatics I: e.g., 1,2,4-thiadiazoles; Cinnamic acids; Melanin biosynthesis inhibitors-reductase (MBI-R): e.g., isobenzofuranone, pyrroloquinolinone, and triazolobenzo-thiazole; Melanin biosynthesis inhibitors-dehydratase (MBI-D): e.g., cyclopropane-carboxamide, carboxamide, and propionamide; Hydroxyanilides (SBI: Class III); Hydroxyanilides (SBI: Class IV): e.g., thiocarbamates and allylamines; Polyoxins: e.g., peptidyl pyrimidine nucleoside; Phenylureas; Quinone inside inhibitors (QiI): e.g., cyanoimidazole and sulfamoyl-triazoles; Benzamides: e.g., toluamides; Antibiotics: e.g., enopyranuronic acid, hexopyranosyl, streptomycin, and validamycin; Cyanoacetamide-oximes; Carbamates; Dinitrophenyl crotonates; Pyrimidinone-hydrazones; 2,6-dinitro-anilines; Organo tin compounds: e.g., tri phenyl tin compounds; Carboxylic acids; Heteroaromatics II: e.g., isoxazoles and isothiazolones; Phosphonates: e.g., ethyl phosphonates and phosphorous acid and salts; Phthalamic acids; Benzotriazines; Benzene-sulfonamides; Pyridazinones; Thiophene-carboxamides; Pyrimidinamides; CAA-fungicides (Carboxylic Acid Amides): e.g., cinnamic acid amides, valinamide carbamates and mandelic acid amides; Tetracycline; Thiocarbamate; Benzamides: e.g., acylpicolides; Host plant defense inducers: e.g., benzo-thiadiazole BTH, benzisothiazole and thiadiazole-carboxamides; Unclassified materials: e.g., thiazole carboxamide, phenyl-acetamide, quinazolinone, and benzophenone; Multi-site contact materials: e.g., copper salts, sulfur, dithiocarbamates and relatives, phthalimides, chloronitriles (phthalonitriles), sulphamides, guanidines, triazines, and quinones (anthraquinones); Non-classified materials: e.g., mineral oils, organic oils, potassium bicarbonate, and biological materials.

Those skilled in the art will recognize that use of other fungicides is also possible in various embodiments of the invention, and failure to list a particular fungicide or fungicidal class herein does not imply limitation of the claims.

The composition of the present invention may be microencapsulated in a polymeric substance. Examples of suitable microencapsulation materials include the following classes of materials for which representative members are provided. It will be apparent to those skilled in the art that other classes of materials with polymeric properties may be used and that other materials within each given class and others polymeric classes may be used for microencapsulation. In this description, microencapsulation is taken to include methods and materials for nanoencapsulation. Examples include but are not limited to: gums and natural macromolecules: such as, gum arabic, agar, sodium alginate, carageenan, and gelatin; carbohydrates: such as, starch, dextran, sucrose, corn syrup, and β-cyclodextrin; celluloses and semisynthetic macromolecules: such as, carboxymethylcellulose, methycellulose, ethylcellulose, nitrocellulose, acetylcellulose, cellulose acetate-phthalate, cellulose acetate-butylate-phthalate, epoxy, and polyester; lipids: such as wax, paraffin, stearic acid, monoglycerides, phospholipids, diglycerides, beeswax, oils, fats, hardened oils, and lechitin; inorganic materials: such as, calcium sulfate, silicates, and clays; proteins: such as, gluten, caseine, gelatine, and albumine; biological materials: such as, voided cells from organisms like baker's yeast and other microorganisms together with other formerly living cell tissues. Furthermore, these materials may be used singly or compounded in the processes of micro- or nano- encapsulation.

Yet another aspect of the present invention is directed to a method of inducing a plant response. This method involves applying to a plant, plant seed, or fruit the composition of the present invention. Application of the composition to a plant, plant seed, or fruit is carried out under conditions effective to induce a plant response to the application of the composition.

The response of plants to the composition includes any response produced by contact with a harpin protein or polypeptide. For example, the response may include, without limitation, disease resistance, plant growth, insect resistance, stress resistance, post-harvest disease resistance, and desiccation resistance.

With regard to imparting disease resistance, absolute immunity against infection may not be conferred, but the severity of the disease may be reduced and symptom development may be delayed. Lesion number, lesion size, and extent of sporulation of fungal pathogens may all be decreased. This method of imparting disease resistance has the potential for treating previously untreatable diseases, treating diseases systemically which might not be treated separately due to cost, and avoiding the use of infectious agents or environmentally harmful materials.

With respect to desiccation, complete protection against desiccation may not be conferred, but the severity of desiccation can be reduced. Desiccation protection inevitably will depend, at least to some extent, on other conditions such as storage temperatures, light exposure, etc. However, controlling desiccation has the potential for eliminating some other treatments (i.e., use of coating waxes) which may contribute to reduced costs or, at least, substantially no increase in costs.

Imparting pathogen resistance to plants in accordance with the present invention is useful in imparting resistance to a wide variety of pathogens including viruses, bacteria, and fungi. Resistance, inter alia, to the following viruses can be achieved by the method of the present invention: Tobacco mosaic virus and Tomato mosaic virus. Resistance, inter alia, to the following bacteria can also be imparted to plants in accordance with present invention: Pseudomonas solancearum, Pseudomonas syringae pv. tabaci, and Xanthamonas campestris pv. pelargonii. Plants can be made resistant, inter alia, to the following fungi by use of the method of the present invention: Fusarium oxysporum and Phytophthora infestans.

With regard to enhancing plant growth, various forms of plant growth enhancement or promotion can be achieved. This can occur as early as when plant growth begins from seeds or later in the life of a plant. For example, plant growth according to the present invention encompasses greater yield, increased quantity of seeds produced, increased percentage of seeds germinated, increased plant size, greater biomass, more and bigger fruit, earlier fruit coloration, and earlier fruit and plant maturation. As a result, compositions of the present invention provide significant economic benefit to growers. For example, early germination and early maturation permit crops to be grown in areas where short growing seasons would otherwise preclude their growth in that locale. Increased percentage of seed germination results in improved crop stands and more efficient seed use. Greater yield, increased size, and enhanced biomass production allow greater revenue generation from a given plot of land.

Insect control according to the present invention encompasses preventing insects from contacting plants to which the hypersensitive response elicitor has been applied, preventing direct insect damage to plants by feeding injury, causing insects to depart from such plants, killing insects proximate to such plants, interfering with insect larval feeding on such plants, preventing insects from colonizing host plants, preventing colonizing insects from releasing phytotoxins, etc. The composition of the present invention may also prevent subsequent disease damage to plants resulting from insect infection.

The composition of the present invention is effective against a wide variety of insects. European corn borer is a major pest of corn (dent and sweet corn) but also feeds on over 200 plant species, including green beans, wax beans, lima beans, soybeans, peppers, potato, tomato, and many weed species. Additional insect larval feeding pests which damage a wide variety of vegetable crops include, without limitation, beet armyworm, cabbage looper, corn ear worm, fall armyworm, diamondback moth, cabbage root maggot, onion maggot, seed corn maggot, pickleworm (melonworm), pepper maggot, and tomato pinworm. Collectively, this group of insect pests represents the most economically important group of pests for vegetable production worldwide.

Another aspect of the present invention is directed to imparting stress resistance to plants. Stress encompasses any environmental factor having an adverse effect on plant physiology and development. Examples of such environmental stress includes, without limitation, climate-related stress (e.g., drought, water, frost, cold temperature, high temperature, excessive light, and insufficient light), air polllution stress (e.g., carbon dioxide, carbon monoxide, sulfur dioxide, NOX, hydrocarbons, ozone, ultraviolet radiation, acidic rain), chemical (e.g., insecticides, fungicides, herbicides, heavy metals), and nutritional stress (e.g., fertilizer, micronutrients, macronutrients). The composition of the present invention may be used to impart resistance to plants against such forms of environmental stress.

This method of the present invention can be used to control a number of postharvest diseases caused by a variety of pathogens. These postharvest diseases and the causative agents which can be treated according to the present invention include, without limitation, the following: Penicillium (e.g., Penicillium digitatum), Botrytis (e.g., Botrytis cinereaon), Phytophthora (e.g., Phytophthora citrophthora), and Erwinia (e.g., Erwinia carotovora).

The method of the present invention involving application of the composition of the present invention can be carried out through a variety of procedures when all or part of the plant is treated including, without limitation, leaves, stems, roots, propagules (e.g., cuttings), fruit, etc. This may (but need not) involve infiltration of the harpin protein or polypeptide into the plant. Suitable application methods include high or low pressure spraying, injection, and leaf abrasion proximate to when elicitor application takes place. Suitable application means may also include atomizing, foaming, fogging, coating, and encrusting.

When treating plant seeds, the harpin protein or polypeptide can be applied by low or high pressure spraying, coating, immersion, or injection. Other suitable application procedures can be envisioned by those skilled in the art provided they are able to effect contact of the hypersensitive response elicitor polypeptide or protein with cells of the plant or plant seed. Once treated with the composition of the present invention, the seeds can be planted in natural or artificial soil and cultivated using conventional procedures to produce plants. After plants have been propagated from seeds treated in accordance with the present invention, the plants may be treated with one or more applications of the composition of the present invention to impart disease resistance to plants, to enhance plant growth, to control insects on the plants, impart stress resistance, and/or post-harvest disease resistance.

Application of the composition of the present invention to a fruit or vegetable may be carried out to enhance the longevity of fruit or vegetable ripeness, as well as inhibit post-harvest disease development in and desiccation of the harvested fruit or vegetable. According to one embodiment, a fruit or vegetable is treated with a liquid composition of the present invention under conditions effective to achieve these effects. Applying a liquid composition of the present invention to a fruit or vegetable can be performed either prior to harvest or after harvest of the fruit or vegetable, using the techniques described herein.

In one embodiment, application of the composition is to a plant. Applying the composition to a plant may be carried out at a rate of about 0.1 to 10,000 g/ha of harpin protein or polypeptide. Preferably, application to a plant is carried out at a rate of about 10 to 1,000 g/ha of harpin protein or polypeptide.

In another embodiment of this method of the present invention, application of the composition is to plant seed. Applying the composition to a plant seed may be carried out at a rate of about 0.001 to 50 g/kg of harpin protein or polypeptide to seed. Preferably, application to a plant seed is carried out at a rate of about 0.01 to 10 g/kg of harpin protein or polypeptide to seed.

The compositions of the present invention can be applied to a plant, plant seed, or fruit in accordance with the present invention alone or in a mixture with other materials. Alternatively, the composition of the present invention can be applied separately to plants with other materials being applied at different times.

The method of the present invention can be utilized to treat a wide variety of plants or their seeds to impart disease resistance, enhance growth, control insects, to impart stress resistance, and/or post-harvest disease resistance. Suitable plants include dicots and monocots. More particularly, useful crop plants can include, without limitation, alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane. Examples of suitable ornamental plants include, without limitation, Arabidopsis thaliana, Saintpaulia, petunia, pelargonium, poinsettia, chrysanthemum, carnation, and zinnia.

These aspects of the present invention are further illustrated by the examples below.

### EXAMPLES

The following examples are provided to illustrate embodiments of the present invention, but they are by no means intended to limit its scope.

### Example 1

### Preparation of a Stable Liquid Composition Containing Harpin αβ and its Efficacy in a Disease Resistance Assay

Recombinant E. coli was used to express harpin αβ (the superharpin of SEQ ID NO: 1) under control of a constitutive promoter. After fermentation, the suspension was diluted in 2-fold volume of potassium phosphate buffer to adjust the pH of the suspension to 7.0.

The resulting solution was heat treated at 95° C. for 10 minutes with a heat exchange system and then cooled within 40 minutes to 45° C. After reaching between about 38-42° C., lysozyme was added to the solution with mixing at the final concentration of 1 ppm, and then allowed to react for 45 minutes. This facilitated breakdown of the bacterial cell wall, resulting in the formation of a crude harpin αβ extract.

The resulting crude extract was then centrifuged for 5 minutes to remove some of the cell debris. The resulting supernatant was divided, and then further heat treated at either (i) 121° C. under 15 psi of pressure for 5 minutes, or (ii) 100° C. for 10 minutes. After the temperature was cooled to 20-25° C., the extract was centrifuged for another 5 -10 minutes at 20,000 rpm to remove the remaining cell debris and some of the denatured proteins. The clarified supernatant, which contained harpin αβ free of cellular debris and most of the denatured proteins, was used to form the stable liquid composition.

In one treatment of both clarified supernatants (i) and (ii), Triple Action disinfectant was added to the harpin αβ extract at the final concentration of 0.5% to further prevent any living organism from growing. In another treatment, neither antibiotics nor disinfectants were added.

The resulting compositions were tested for (i) harpin αβ protein with HPLC analysis and (ii) its efficacy in a disease resistance assay. A comparably processed composition containing phosphate buffered saline with and without 0.5% Triple Action were used as negative controls. The commercial product ProActm (Plant Healthcare, Inc.) with 1% harpin αβ was used as a positive control in the disease resistance induction assay.

HPLC Analysis

For the HPLC analysis the following tests were conducted. Sample 1: 100° C. treated for 10 mintues without Triple Action; Sample 2: 100° C. treated for 10 minutes plus 0.5% Triple Action; Sample 3: 121° C. treated for 5 mintues without Triple Action; and Sample 4: 121° C. treated for 5 mintues with 0.5% Triple Action.

HPLC analysis indicated that liquid harpin αβ composition is only stable for about 4 weeks in the treatment of 100° C. for 10 min without the addition of a disinfectant. The same treatment, but with the addition of 0.5% Triple Action disinfectant resulted in harpin αβ stability for more than 3 months. The treatment at 121° C. for 5 min with or without addition of the disinfectant is also stable for more than 3 months. The results of these experiments showing the concentration of harpin αβ are set forth in Table 4 below.

TABLE 4
Stability Data of Liquid Compositions

| | Week | | | | | | |
|---|---|---|---|---|---|---|---|
| | 1 | 2 | 3 | 4 | 5 | 6 | 7 |
| 100° C., 10 min., no disinfectant | 5.87 | 6.35 | 6.32 | 4.5 | 4.7 | 4.3 | 3.8 |
| 100° C., 10 min., 0.5% | 5.61 | 5.97 | 5.51 | 6.02 | 6 | 6.6 | 6.1 |
| disinfectant (Triple Action) | | | | | | | |
| 121° C., 5 min., no disinfectant | 5.67 | 6.6 | 6.4 | 5.6 | 5.4 | 5.4 | 5.9 |
| 121° C., 5 min., 0.5% | 6.98 | 6.2 | 6.6 | 5.7 | 6.4 | 6.4 | 5.89 |
| disinfectant (Triple Action) | | | | | | | |

| | Week | | | | | | | |
|---|---|---|---|---|---|---|---|---|
| | 8 | 9 | 10 | 11 | 12 | 13 | 14 | 15 |
| 100° C., 10 min., no disinfectant | 2.7 | — | — | — | — | — | — | — |
| 100° C., 10 min., 0.5% | 5.9 | 5.7 | 5.86 | 5.95 | 5.7 | 5.65 | 6.23 | 5.71 |
| disinfectant (Triple Action) | | | | | | | | |
| 121° C., 5 min., no disinfectant | 5.88 | 5.75 | 5.5 | 5.4 | 5.6 | 5.5 | 5.7 | 5.65 |
| 121° C., 5 min., 0.5% | 6.5 | 6.6 | 6.6 | 6.7 | 6.2 | 5.57 | 5.4 | 6.1 |
| disinfectant (Triple Action) | | | | | | | | |

These results indicate that biodegradation of a liquid formulation of harpin αβ protein is primarily attributed to the natural environment of a living organism. However, with high temperature treatment or in the presence of a disinfectant the liquid formulation of harpin αβ protein can remain stable for an extended period of time.

Disease Resistance Induction Tests

Disease resistance induction tests were conducted as follows. In addition to the four samples described above, a 1% dry product ProAct™ was used as a positive control and 5 mM potassium phosphate buffer with and without disenfectant were used as negative controls. All of the harpin containing samples were topically applied by foliar spray at a rate of 5 ppm to 6 tobacco seedlings 8 weeks following emergence of the first true leaves. All seedlings were grown in greenhouse conditions at a day temperature of about 25° C. and a night temperature of about 18° C., with about 16 hours of sunlight during the day and about 8 hours of darkness at night. Humidity was kept at around 70%.

The tobacco plants were challenged with a tobacco mosaic virus (“TMV”) at a concentration of 2 μg/ml. Disease severity was assessed by counting the number of lesions appearing on the leaves 7 days after the inoculation. The test data (Table 5) showed that the liquid harpin αβ compositions achieved comparable results to the commercial product ProAct™ in terms of reduction of TMV lesions. Given the substantial benefits offered by liquid formulations over the powdered formulations, noted above, users should prefer the liquid formulations given the comparable efficacy.

TABLE 5
Disease Resistance Induction Test Results

| | Plant | | | | | | | % Reduction |
|---|---|---|---|---|---|---|---|---|
| | 1 | 2 | 3 | 4 | 5 | 6 | Average | vs. Control |
| 100° C., 10 min., | 11 | 17 | 21 | 5 | 9 | 18 | 14 | 75 |
| no disinfectant | | | | | | | | |
| 100° C., 10 min., | 7 | 19 | 26 | 19 | 4 | 7 | 14 | 74 |
| 0.5% disinfectant | | | | | | | | |
| (Triple Action) | | | | | | | | |
| 121° C., 5 min., no | 15 | 13 | 9 | 21 | 3 | 18 | 13 | 77 |
| disinfectant | | | | | | | | |
| 121° C., 5 min., | 17 | 23 | 33 | 11 | 12 | 19 | 19 | 61 |
| 0.5% disinfectant | | | | | | | | |
| (Triple Action) | | | | | | | | |
| ProAct ™ with | 16 | 3 | 28 | 28 | 11 | 12 | 16 | 70 |
| 1% harpin αβ | | | | | | | | |
| protein | | | | | | | | |
| 5 mM potassium | 81 | 67 | 72 | 50 | 54 | 87 | 69 | — |
| phosphate buffer | | | | | | | | |
| 5 mM potassium | 76 | 89 | 54 | 62 | 71 | 58 | 68 | — |
| phosphate buffer | | | | | | | | |
| with 0.5% | | | | | | | | |
| disinfectant | | | | | | | | |
| (Triple Action) | | | | | | | | |

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of making a stable liquid composition containing a harpin protein or polypeptide, the method comprising:
obtaining a liquid extract that is substantially free of cellular debris and comprises a harpin protein or polypeptide; and
introducing into the liquid extract a biocidal agent and, optionally, one or both of a protease inhibitor and a non-ionic surfactant, thereby obtaining a liquid composition comprising the harpin protein or polypeptide that retains harpin activity for at least about 72 hours.

2. The method according to claim 1, wherein the harpin protein or polypeptide is selected from one or more of the group consisting of homologs of Erwinia amylovora HrpN, homologs of Erwinia amylovora HrpW, homologs of Pseudomonas syringae HrpW, homologs of Pseudomonas syringae HrpZ, homologs of Xanthomonas campestris HreX, and a fusion protein comprising two or more hypersensitive response eliciting domains.

3. The method according to claim 1, wherein said obtaining comprises:
heating a fermented bacterial suspension to a temperature of about 60 to 100° C.;
cooling the suspension;
lysing cells in the bacterial suspension; and
removing cellular debris from the suspension.

4. The method according to claim 3, further comprising, prior to said heating, adjusting the pH of the fermented bacterial suspension to a pH of about 5.5 to 10.

5 . The method according to claim 4, wherein said adjusting is carried out using a potassium phosphate buffer or a Tris-EDTA buffer.

6. The method according to claim 3, wherein said lysing is carried out with a lysozyme at a concentration of about 1 ppm to 100 ppm.

7. The method according to claim 1, wherein the biocidal agent is introduced into the liquid extract at a concentration of about 1 ppm to 100 ppm.

8. The method according to claim 1, wherein a protease inhibitor is introduced into the liquid extract.

9. The method according to claim 8, wherein the protease inhibitor is introduced into the liquid extract at a concentration of about 1 ppm to 100 ppm.

10. The method according to claim 8, wherein the protease inhibitor is selected from aprotinin, bestatin, calpain inhibitor I, calpain inhibitor II, chymostatin, E-64, leupeptin, alpha-2-macroglobuline, pefabloc SC, pepstatin, phenylmethanesulfonyl fluoride, and tosyl-L-lysine chloromethyl ketone.

11. The method according to claim 1, wherein a non-ionic surfactant is introduced into the liquid extract.

12. The method according to claim 11, wherein the non-ionic surfactant is introduced into the liquid extract at a volume amount of about 0.05% to about 10%.

13. The method according to claim 1, wherein both a protease inhibitor and a non-ionic surfactant are introduced into the liquid extract.

14. A liquid composition obtained by the method of claim 1.

15. A composition comprising an aqueous carrier, a harpin protein or polypeptide, an effective amount of a biocidal agent, and optionally, an effective amount of one or both of a protease inhibitor and a non-ionic surfactant, whereby the composition retains harpin activity for at least about 72 hours.

16. The composition according to claim 15, wherein the harpin protein or polypeptide is selected from one or more of the group consisting of homologs of Erwinia amylovora HrpN, homologs of Erwinia amylovora HrpW, homologs of Pseudomonas syringae HrpW, homologs of Pseudomonas syringae HrpZ, homologs of Xanthomonas campestris HreX, and a fusion protein comprising two or more hypersensitive response eliciting domains.

17. The composition according to claim 15, wherein the biocidal agent is present in an amount of about 1 ppm to 100 ppm.

18. The composition according to claim 15 in the form of a solution, emulsion, emulsifiable concentrate, suspension, foam, aerosol, suspoemulsion concentrate, slurry, or paste.

19. The composition according to claim 15 further comprising:
an effective amount of an herbicide, insecticide, attractant, sterilant, bactericide, acaricide, nematicide, fungicide, and/or growth regulator.

20. The composition according to claim 15, wherein the composition is microencapsulated in a polymeric substance.

21. The composition according to claim 15, wherein the composition retains at least about 50% activity for more than 72 hours.

22. The composition according to claim 15, wherein the composition retains at least about 90% activity for more than 3 months.

23. The composition according to claim 15, comprising a protease inhibitor.

24. The composition according to claim 23, wherein the protease inhibitor is present in an amount of about 1 ppm to 100 ppm.

25. The composition according to claim 23, wherein the protease inhibitor is selected from aprotinin, bestatin, calpain inhibitor I, calpain inhibitor II, chymostatin, E-64, leupeptin, alpha-2-macroglobuline, pefabloc SC, pepstatin, PMSF, and TLCK.

26. The composition according to claim 15, comprising a non-ionic surfactant.

27. The composition according to claim 26, wherein the non-ionic surfactant is present in a volume amount of about 0.05% to about 10%.

28. The composition according to claim 15, comprising both a protease inhibitor and a non-ionic surfactant.

29. A method of inducing a plant response comprising:
applying to a plant, plant seed, or fruit the composition according to claim 14, said applying being carried out under conditions effective to induce a plant response to said applying.

30. The method according to claim 29, wherein the plant response is one or more of disease resistance, plant growth, insect resistance, and desiccation resistance.

31. The method according to claim 29, wherein said applying is carried out by spraying, atomizing, foaming, fogging, coating, and/or encrusting.

32. The method according to claim 29, wherein said applying is to a plant at a rate of about 0.1 to 10,000 g/ha of harpin protein or polypeptide.

33. The method according to claim 29, wherein said applying is to a plant seed at a rate of about 0.001 to 50 g/kg of harpin protein or polypeptide to seed.


SEQUENCE LISTING (2 sequences)

SEQ ID NO: 1 - protein, 412 aa, Artificial Sequence
  Superharpin alpha-beta
MSLNTSGLGASTMQISIGGAGGNNGLLGTHMPGTSSSPGLFQSGGDNGLGGHNANSALGQQPIDRQTIEQMAQLLAELLKSLLDSGEKLGDNFGASADSASGTGQQDLMTQVLNGLAKSMLDDLLTKQDGGTSFSEDDSGPAKDGNANAGANDPSKNDPSKSQGPQSANKTGNVDDANNQDPMQALMQLLEDLVKLLKAALHMQQPGGNDKGNGVGGDSGQNDDSTSGTDSTSDSSDPMQQLLKMFSEIMQSLFGDEQDGTDSTSGSRFTRTGIGMKAGIQALNDIGTHSDSSTRSFVNKGDRAMAKEIGQFMDQYPEVFGKPQYQKGPGQEVKTDDKSWAKALSKPDDDGMTPASMEQFNKAKGMIKSAMAGDTGNGNLQARGAGGSSLGIDAMMAGDAINNMALGKLGAA

SEQ ID NO: 2 - DNA, 1239 bp, Artificial Sequence
  Superharpin alpha-beta
atgagtctgaatacaagtgggctgggagcgtcaacgatgcaaatttctatcggcggtgcgggcggaaataacgggttgctgggtacgcatatgcccgggacctcgtcctcgccgggtctgttccagtccgggggggacaacgggcttggtggtcataatgcaaattctgcgttggggcaacaacccatcgatcggcaaaccattgagcaaatggctcaattattggcggaactgttaaagtcactgctagatagtggggaaaagctcggtgacaacttcggcgcgtctgcggacagcgcctcgggtaccggacagcaggacctgatgactcaggtgctcaatggcctggccaagtcgatgctcgatgatcttctgaccaagcaggatggcgggaccagcttctccgaagacgatagtgggccggcgaaggacggcaatgccaacgcgggcgccaacgacccgagcaagaacgacccgagcaagagccagggtccgcagtcggccaacaagaccggcaacgtcgacgacgccaacaaccaggatccgatgcaagcgctgatgcagctgctggaagacctggtgaagctgctgaaggcggccctgcacatgcagcagcccggcggcaatgacaagggcaacggcgtgggcggtgatagtgggcaaaacgacgattccacctccggcacagattccacctcagactccagcgacccgatgcagcagctgctgaagatgttcagcgagataatgcaaagcctgtttggtgatgagcaagatggcaccgatagtactagcggctcgaggtttactcgtaccggtatcggtatgaaagcgggcattcaggcgctgaatgatatcggtacgcacagcgacagttcaacccgttctttcgtcaataaaggcgatcgggcgatggcgaaggaaatcggtcagttcatggaccagtatcctgaggtgtttggcaagccgcagtaccagaaaggcccgggtcaggaggtgaaaaccgatgacaaatcatgggcaaaagcactgagcaagccagatgacgacggaatgacaccagccagtatggagcagttcaacaaagccaagggcatgatcaaaagcgccatggcgggtgataccggcaacggcaacctgcaggcacgcggtgccggtggttcttcgctgggtattgatgccatgatggccggtgatgccattaacaatatggcacttggcaagctgggcgcggcttaa